(19) United States
(12) Patent Application Publication
(10) Pub. No.: US 2019/0314438 A1
(43) Pub. Date: Oct. 17, 2019

(54) ORGANIC EYE DROPS

(71) Applicant: Eyeganics LLC, Kirkwood, MO (US)

(72) Inventor: John Daniel Friederich, Kirkwood, MO (US)

(21) Appl. No.: 16/295,901

(22) Filed: Mar. 7, 2019

Related U.S. Application Data
(60) Provisional application No. 62/658,456, filed on Apr. 16, 2018.

(51) Int. Cl. A61K 36/64; A61K 35/06; A61K 31/047; A61K 47/10; A61K 47/12; A61K 9/00; A61P 27/02

(52) U.S. Cl. CPC A61K 36/64; A61K 35/06; A61K 31/047; A61P 27/02; A61K 47/12; A61K 9/0048; A61K 47/10

(57) ABSTRACT

Organic eye drops are formulated and produced to adhere simultaneously to federal current good manufacturing practices (CGMP) for drugs and pharmaceuticals, the over-the-counter (OTC) eye drop monograph, and the National Organic Program. The first step in making an organic eye drop is finding an active ingredient that is (1) pharmaceutical grade (USP); (2) certified organic; and (3) suitable for use as an eye drop active ingredient. After sourcing an active ingredient that meets the three criteria identified in the first step, above, a suitable eye drop manufacturer is necessary. The manufacturer's facility should be (1) certified organic; and (2) in compliance with federal drug manufacturing regulations. In manufacturing, prohibited substances are prevented from commingling with the eye drops from beginning to end.

### CROSS-REFERENCE TO RELATED APPLICATIONS

This application claims the benefit of priority to U.S. provisional patent application No. 62/658,456, filed Apr. 16, 2018, the contents of which are hereby incorporated by reference.

### BACKGROUND OF THE INVENTION

### 1. Field of the Invention

One or more embodiments of the invention relates generally to eye drops. More particularly, the invention relates to an organic eye drop that can provide relief of ocular symptoms with an over-the-counter drop produced in compliance with organic standards.

### 2. Description of Prior Art and Related Information

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

While there is a wide selection of over-the-counter (OTC) eye drops available for the temporary relief of myriad ocular symptoms, no eye drop exists that is certified organic. There is growing global demand for organic products and, while many organic innovations have arrived across a wide range of disciplines, the field of eye care has no organic eye drops.

OTC eye drop manufacturing is a strictly-regulated process requiring adherence to current good manufacturing practices for drugs and finished pharmaceuticals; OTC eye drops also have to follow the OTC ophthalmic drug monograph regulations. Likewise, organic production requires careful compliance with specific federal organic standards. This invention, organic eye drops, creatively adheres to each of the applicable federal rules by using an innovative formulation and production process.

In view of the foregoing, there is a need for an organic eye drop and methods for producing the same.

### SUMMARY OF THE INVENTION

Organic eye drops fill an enormous void in the eye care industry, providing relief of ocular symptoms with an OTC drop produced in compliance with organic standards. The invention innovatively combines the current criteria of OTC eye drop manufacturing with organic ingredients and processes.

According to an exemplary embodiment of the present invention, an organic eye drop comprises an active ingredient, wherein the active ingredient is pharmaceutical grade, certified organic, and suitable for use as an eye drop active ingredient; and one or more inactive ingredients, wherein the one or more inactive ingredients are at least one or certified organic and listed on a list of allowed substances list of the national organic program.

In some embodiments, the active ingredient is listed on at least one of the over-the-counter eye drop monograph of the Food and Drug Administration and the Homeopathic Pharmacopoeia of the United States.

In some embodiments, the active ingredient is selected the group consisting of gelatin, glycerin, mineral oil, lanolin, Euphrasia, and combinations thereof.

In some embodiments, the one or more inactive ingredients is selected from the group consisting of vehicle, adjuvants, preservatives, buffering agents and viscosity agents.

In some embodiments, the preservative is citric acid.

In some embodiments, the buffering agent is selected from the group consisting of sodium citrate, glycerin and potassium citrate.

In some embodiments, the eye drops are free from preservatives.

In some embodiments, the organic eye drops are over-the-counter organic eye drops.

According to another exemplary embodiment, the present invention provides an organic eye drop comprising an active ingredient, wherein the active ingredient is pharmaceutical grade, certified organic, and listed on least one of the over-the-counter eye drop monograph of the Food and Drug Administration and the Homeopathic Pharmacopoeia of the United States; and one or more inactive ingredients, wherein the one or more inactive ingredients are at least one or certified organic and listed on a list of allowed substances list of the national organic program and the one or more inactive ingredients is selected from the group consisting of vehicle, adjuvants, preservatives, buffering agents and viscosity agents.

According to another exemplary embodiment, the present invention provides a method for the preparation of an over-the-counter organic eye drops, the method comprising formulating an active ingredient with one or more inactive ingredients to form an eye drop formulation wherein the active ingredient pharmaceutical grade, certified organic, suitable for use as an eye drop active ingredient, the eye drop formulation is prepared in a manufacturing facility that is certified organic and in compliance with federal drug manufacturing regulations, and the one or more inactive ingredients are at least one of certified organic and on the allowed substances list of the national organic program; and sterilizing the eye drop formulation via at least one of filtration and heating.

These and other features, aspects and advantages of the present invention will become better understood with reference to the following drawings, description and claims.

### DETAILED DESCRIPTION OF THE PREFERRED EMBODIMENTS AND BEST MODE OF INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details.

As is well known to those skilled in the art, many careful considerations and compromises typically must be made when designing for the optimal configuration of a commercial implementation of any system, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Organic eye drops are formulated and produced in a never-before-seen way. They simultaneously adhere to federal current good manufacturing practices (CGMP) for drugs and pharmaceuticals, the over-the-counter (OTC) eye drop monograph, and the National Organic Program.

The first step in making an organic eye drop is finding an active ingredient that is (1) pharmaceutical grade (USP); (2) certified organic; and (3) suitable for use as an eye drop active ingredient. As used herein, ingredients “suitable for use as an eye drop active ingredient” can include ingredients that are typically used in eye drops, such as gelatin, glycerin, mineral oil, lanolin, Euphrasia, polyethylene glycol, polypropylene glycol, carboxymethylcellulose sodium, povidone, polyvinyl alcohol, polysorbate, white petroleum, Hypromellose, dextran, polyvinyl pyrrolidone, and the like. In should be understood that this list of ingredients “suitable for use as an eye drop active ingredient” is not exhaustive and may or may not be usable in the organic eye drop composition of the present invention, as some of these compounds may not qualify with the first two requirements (pharmaceutical grade and certified organic). In some embodiments, these ingredients suitable for use as an eye drop active ingredient may be listed on at least one of the OTC eye drop monograph and the Homeopathic Pharmacopoeia of the United States.

There are only four active ingredients listed on the OTC eye drop monograph that can possibly be produced organically and certified as organic; the remainder of the active ingredients listed are inorganic chemicals. The four active ingredients with organic potential are: gelatin, glycerin, mineral oil, and lanolin. In addition to the OTC eye drop monograph of the FDA, the Homeopathic Pharmacopoeia of the United States (HPUS) contains dozens of “Official Homeopathic Drugs” that can be produced organically and used as active ingredients in eye drops. While there are specific homeopathic procedures in place to ensure that these active ingredients are produced according to the HPUS, finished drug products' CGMP are still ultimately regulated by the FDA. For example, euphrasia is a plant-based homeopathic drug ingredient that can be sourced organically and then used in homeopathic eye drops.

After sourcing an active ingredient that meets the three criteria identified in the first step, above, a suitable eye drop manufacturer is necessary. The manufacturer's facility has to be (1) certified organic; and (2) in compliance with federal drug manufacturing regulations.

The requirements for the sterile manufacturing of eye drops are meticulous, extensive and well-established; these manufacturing facilities are regularly inspected by the Food and Drug Administration (FDA) in order to be allowed to produce eye drops. Having one's pharmaceutical facility certified by an agent of the National Organic Program (NOP) is significantly less cumbersome than meeting the stringent drug manufacturing requirements of the FDA. Nonetheless, there are facility changes that may be required in order to meet the requirements of these separate agencies depending on the specific operations of the facility.

With an active ingredient and a suitable manufacturer lined up, one can now formulate the eye drop. Any inactive ingredients (vehicle, adjuvants, preservatives, buffering agents, and viscosity agents) included in the eye drop formulation must be certified organic and/or on the allowed substances list of the NOP.

Most multi-use eye drop bottles require a preservative, and the majority of the eye drop preservatives allowed by the FDA are inorganic chemicals. The NOP's allowed substances list does include some ingredients that could theoretically be used as an eye drop preservative. Citric acid, for example, is allowed by the NOP as a processing aid, and is generally recognized as safe (GRAS) by the FDA. Eye drops can also be manufactured into single-use preservative-free vials or multi-use preservative-free eye drop bottles. There are many preservative-free eye drops commercially available both in single-use and multi-use systems.

Buffering agents and viscosity agents are not technically required per FDA regulations, though they are instrumental to balance an eye drop's pH and keep the eye drop stable in the eye. The NOP's allowed substances list includes some ingredients that could theoretically be used as eye drop buffering agents or viscosity agents. These include but are not limited to sodium citrate, glycerin, and potassium citrate.

With the eye drop's active and inactive ingredients formulated, the manufacturing process can begin. The key to organically manufacturing an eye drop is to prevent prohibited substances from commingling with the drop from beginning to end. Sterility is the primary focus of the FDA's CGMP for eye drop manufacturing, with complete sterility required from start to finish; the majority of cleaners, sanitizers, and processing aids commonly used in eye drop manufacturing are prohibited by organic standards. Any sanitizer residues that will be left on the manufacturing equipment must be on the NOP's allowed substances list. Section 205.605 of the National List explicitly mentions the following allowed sanitizers: acidified sodium chlorite, chlorine materials (including bleach), hydrogen peroxide, ozone, and peracetic acid/peroxyacetic acid. Phosphoric acid is the only cleaner on the National List. All other cleaners must be fully rinsed from drug-contact surfaces prior to production. A manufacturer can dedicate equipment to organic manufacturing or can use a cleaning step (with an allowed cleaner and sanitizer) between nonorganic and organic production batches. As far as the manufacturing process itself, sterilization of the eye drop must be achieved without the use of any prohibited substances. After the sterile solution is prepared to volume, the solution is then clarified, filled into final containers, sterilized, and sealed. Eye drop sterilization is typically achieved through filtration or heating. Filtration works well for sterilization, as it uses no chemicals. Heating typically uses benzalkonium chloride or other prohibited substances.

While all of the above steps reference processes and ingredients that have been readily available, the innovation is the creative and unique combination of ingredients and processes being utilized; this has never been accomplished.

[Dan—do we have any specific formulations we can insert here as specific examples of the eye drop formulation? We will not limit our broadest claims to any specifics, but it could be helpful. Also, could we provide concentration ranges in the formulation that may be beneficial? For example, from about 0.1% to about 1% by weight glycerin, or from about 1% to about 20% mineral oil?]

The organic over-the-counter eye drop formulation of the present invention may be useful for the treatment and/or prevention of issues with the eye, including itchy eyes, dry eye, burning eyes, irritated eyes, or the like. In some embodiments, the eye drop formulation of the present invention may include one or more additional active ingredients that may include eye numbing agents, allergy treatment agents, antibiotics, corticosteroids, antivirals, anti-inflammatories and the like. The organic over-the-counter eye drop formulation may be used in the treatment and/or prevention of these, or other known conditions of the eye, by administering a therapeutically effective amount of the eye drop to a patient in need thereof, by dropping one or more doses of the eye drops into the eye of the patient.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Claim elements and steps herein may have been numbered and/or lettered solely as an aid in readability and understanding. Any such numbering and lettering in itself is not intended to and should not be taken to indicate the ordering of elements and/or steps in the claims.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what incorporates the essential idea of the invention.

What is claimed is:

1. An organic eye drop comprising:
an active ingredient, wherein the active ingredient is pharmaceutical grade, certified organic, and suitable for use as an eye drop active ingredient; and
one or more inactive ingredients, wherein the one or more inactive ingredients are at least one or certified organic and listed on a list of allowed substances list of the national organic program.

2. The organic eye drop of claim 1, wherein the active ingredient is listed on at least one of the over-the-counter eye drop monograph of the Food and Drug Administration and the Homeopathic Pharmacopoeia of the United States.

3. The organic eye drop of claim 1, wherein the active ingredient is selected the group consisting of gelatin, glycerin, mineral oil, lanolin, Euphrasia, and combinations thereof.

4. The organic eye drop of claim 1, wherein the one or more inactive ingredients is selected from the group consisting of vehicle, adjuvants, preservatives, buffering agents and viscosity agents.

5. The organic eye drop of claim 4, wherein the preservative is citric acid.

6. The organic eye drop of claim 4, wherein the buffering agent is selected from the group consisting of sodium citrate, glycerin and potassium citrate.

7. The organic eye drop of claim 1, wherein the eye drops are free from preservatives.

8. The organic eye drop of claim 1, wherein the organic eye drop is an over-the-counter organic eye drop.

9. An organic eye drop comprising:
an active ingredient, wherein the active ingredient is pharmaceutical grade, certified organic, and listed on least one of the over-the-counter eye drop monograph of the Food and Drug Administration and the Homeopathic Pharmacopoeia of the United States; and
one or more inactive ingredients, wherein
the one or more inactive ingredients are at least one or certified organic and listed on a list of allowed substances list of the national organic program; and
the one or more inactive ingredients is selected from the group consisting of vehicle, adjuvants, preservatives, buffering agents and viscosity agents.

10. The organic eye drop of claim 9, wherein the active ingredient is selected the group consisting of gelatin, glycerin, mineral oil, lanolin, Euphrasia, and combinations thereof.

11. The organic eye drop of claim 9, wherein the preservative is citric acid.

12. The organic eye drop of claim 9, wherein the buffering agent is selected from the group consisting of sodium citrate, glycerin and potassium citrate.

13. The organic eye drops of claim 9, wherein the eye drops are free from preservatives.

14. A method for the preparation of an over-the-counter organic eye drops, comprising:
formulating an active ingredient with one or more inactive ingredients to form an eye drop formulation wherein:
the active ingredient pharmaceutical grade, certified organic, suitable for use as an eye drop active ingredient,
the eye drop formulation is prepared in a manufacturing facility that is certified organic and in compliance with federal drug manufacturing regulations, and
the one or more inactive ingredients are at least one of certified organic and on the allowed substances list of the national organic program;
sterilizing the eye drop formulation via at least one of filtration and heating.

15. The method of claim 14, wherein the active ingredient is listed on at least one of the over-the-counter eye drop monograph of the Food and Drug Administration and the Homeopathic Pharmacopoeia of the United States.

16. The method of claim 14, wherein the active ingredient is selected the group consisting of gelatin, glycerin, mineral oil, lanolin, Euphrasia, and combinations thereof.

17. The method of claim 14, wherein the one or more inactive ingredients is selected from the group consisting of vehicle, adjuvants, preservatives, buffering agents and viscosity agents.
